(19) United States
(12) Patent Application Publication
(10) Pub. No.: US 2010/0145158 A1
(43) Pub. Date: Jun. 10, 2010

(54) Pod Connected Data Monitoring System

(76) Inventors: Scott E. Hamilton, Los Angeles, CA (US); Robin Ross Cooper, La Mesa, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 12/083,115

(22) PCT Filed: Oct. 6, 2006
(86) PCT No.: PCT/US06/39280

§ 371 Date: Apr. 4, 2008

Related U.S. Application Data
(60) Provisional application No. 60/724,150, filed on Oct. 6, 2005.

(51) Int. Cl. A61B 5/00; H04N 7/16; H04Q 5/22; G06Q 50/00

(52) U.S. Cl. 600/300; 725/14; 340/10.1; 705/3

(57) ABSTRACT

In a perfect world, citizens would have all of the sensing equipment and analysis capability with no concern about costs. Unfortunately, the costs are an impediment to most people and their health and well-being may be compromised as a result. By leveraging popular consumer devices such as mobile players and other CE devices, it is possible to create “piggy-back” solutions that effectively bring the costs into the range that ordinary people can afford. Once ordinary people can afford these solutions, the state of their health and/or well being can be improved greatly.

### SUMMARY OF THE INVENTION

System and network that leverages the popularity and functionality of mobile content playback devices for the purpose of capturing real-time data points for immediate or future analysis and potential intervention.

### BACKGROUND OF THE INVENTION

In the past, significantly large groups of people have had the need to capture data points in real-time for the purpose of analyzing such data for potential risks or threats. For example, a patient who has recently experienced the failure of a vital organ may need to continuously collect and analyze vital statistics in order to be assured that he or she is not in any imminent danger. The problem has been the prohibitive cost of such a system. If a company were to architect and manufacture a device that would provide them with information about their own vital statistics, it would most likely be unmarketable as it would be too expensive for the intended population.

From time to time, technologies are introduced to the market that are not intended to solve health problems, however, they provide effective building blocks that can be adapted and modified to ultimately assist people in maintaining their health. When these technologies reach a critical mass in the market place, they can then be used as a platform to introduce new and important solutions by allowing a leveraging or “piggy-back” effect based on the success of the original product.

The intention of this patent application is to teach a method and a utility for leveraging the use of a consumer's iPod™ device manufactured by Apple Computer, Inc.™ (or other similar devices) in order to capture data points in real-time for the purposes of analyzing such data and effectively reporting the results back to the consumer.

### Overview of the Invention

In the preferred embodiment, a patient with a health risk may wish to use his or her Apple iPod™ device to collect vital statistics during the course of an hour, day, week, month, or year. The memory capacity within the iPod™ can be used to hold the data points as they are being collected. At an appropriate time, the patient can then plug the iPod© into a data analysis facility so the data points can be effectively analyzed and the appropriate recommendations can be sent back to the patient. Such analysis can be performed by plugging the iPod™ into another consumer electronics (CE) device such as a PC or a common television set-top-box. With an appropriate harness or cradle, the data points from the iPod™ can be transmitted to a more powerful CE device that has network connectivity. Once these data have been effectively analyzed, the patient can receive instructions regarding how best to proceed on the path to improved health. Examples of instructions can be (but are not limited to) the following:
Text Messages on a mobile device
Printouts on a printer (wireless or fixed line)
Messages on a TV screen
Messages on the iPod™ itself
Other information platforms
Care givers

The important thing to note is the way that a number of popular consumer oriented devices can be leveraged in order to create a low-cost solution for monitoring and reporting data points.

Note: For the purposes of this patent application, the iPod™ will now be referred to simply as a “Pod”.

### General (Simplified) System Architecture

The current state-of-the-art pods that exist today for either music, or video, or both are typically designed with connectors both on the top of the unit and at the bottom of the unit, or either or both sides. In the preferred embodiment, either a clam-shell style adapter would wrap itself around the Pod in such a way as to connect to both the top connectors and the bottom connectors simultaneously, or adapters could be stacked atop the pod in a tower fashion. In addition, this clam shell or tower adapter houses the extra logic, sensors, and software that capture the desired data elements. As these data elements are being captured, the adapters feed data to the Pod with instructions regarding “where” and “how” to store these data elements. Given the vast amount of memory available to a current-day Pod, large amounts of data can be stored with little concern about “running out of on-board system resources”.

A companion harness or cradle (from now on referred to as the cradle) can then be used to hold the Pod while it gains access to the stored data elements and then transfers them securely to a network for analyses and reporting purposes. For example, the data can be analyzed by a physician connected to the network or by a device connected to the network.

One embodiment of the system is depicted below:

Sensors→Pod→Cradle→CE device→Network→Reporting Mechanism

### The Major Benefits of a Pod Device

The best things about leveraging a Pod device for the purposes mentioned above are:
It's built to be low-cost
It is a ubiquitous product
Simple user interface
It has the necessary storage, processing, and communications capability
It can receive information from data-collection devices
It can store large amounts of data easily and efficiently
It has numerous other advantages as well.

### BRIEF DESCRIPTION OF DRAWINGS

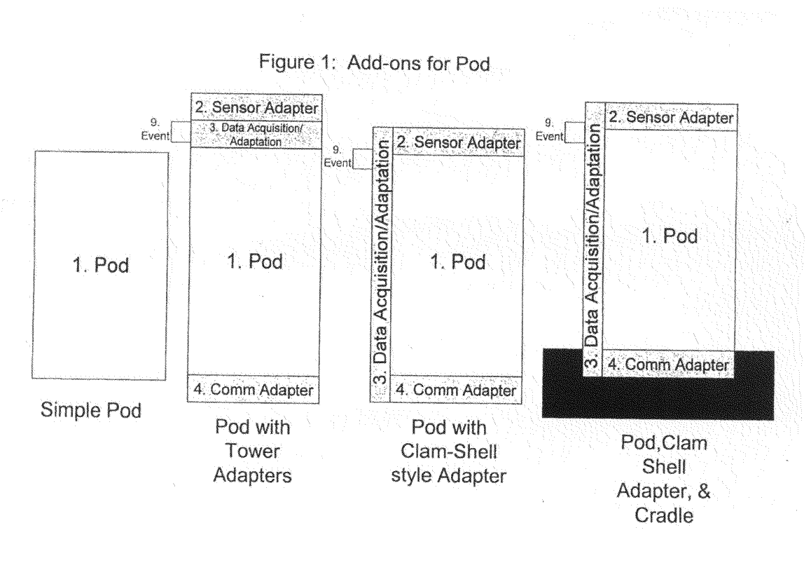

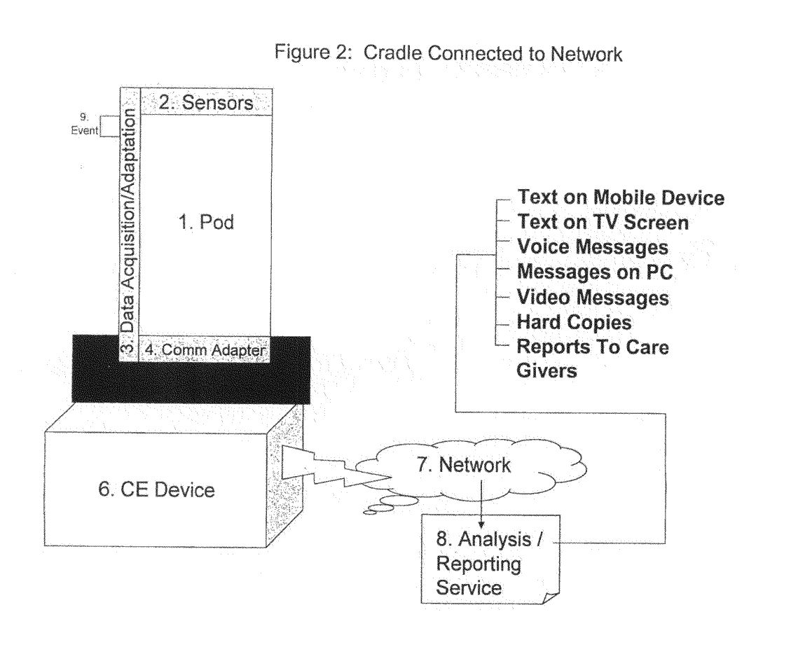

FIG. 2 presents a high-level view of the system. The component parts are as follows: The Pod (1) refers to any portable player device such as the Apple iPod™ or similar device. Such a Pod (1) does not necessarily need to be a “music-only” device. Such a device can be dedicated to the following:
Still images
Documents
Video
Other data elements (digitized analog, digital, or both).

Additionally, such a Pod can be manufactured or distributed by virtually any company as long as the device has either achieved a minimal level of success, or such a device has the potential to achieve a minimal level of success and supports essentially the same functional capabilities of the iPod™: digital input/output, data storage, internal battery, text/video screen, user interface controls.

The Sensor Adapter (2) is the module that interfaces to sensing devices that capture the empirical data in real-time.

The Data Acquisition/Adaptation Module (3) collects the empirical data received by the Sensor Adapter (2) and prepares the data in such a way that it can be communicated to the Pod for storage and subsequent external transmittal. Additionally, an Event Input (9) is used to indicate that a significant event has occurred that should be noted in the data or that signals a trigger event to the set-top-box via a short range radio frequency signal.

The Communications Adapter (4) is the input/output device that links the Pod (1) to the outside world. The primary function for this Communications Adapter (4) is to send the data elements that were captured by the Sensor Adapter (2) and prepared by the Data Acquisition/Adaptation Module (3) then subsequently stored on the Pod (1) to the Network (7) in order for the consumer to eventually receive his or her Analysis Reports (8). It is important to note that the Communications Adapter (4) is a two-way device.

Although its primary function is to send data elements to the outside world, it can also receive data for a number of purposes such as:
Updating the software in the Pod (1)
Updating the software in the Sensor Adapter (2)
Updating the software in the Data Acquisition/Adaptation Adapter (3)
Presenting information on the Pod's screen
Sending instructions or requests to any of the elements mentioned above.

In one embodiment the Cradle (5) is not a part of the Pod (1). The Cradle (5) is an extension of a popular CE Device (6) such as a PC or a set-top-box. This Cradle (5) facilitates the coupling of the Pod (1) to the CE Device (6). Once the Pod (1) is connected to the Cradle (5) by way of the Communications Adapter (4), then the flow of information from the Pod to the CE Device (6) (and vice-versa), can take place.

The CE Device (6) is the component that has the extra system resources required to take the data points received from the Pod (1) and send them through the appropriate network for analyses. Although the primary purpose for the CE Device (6) is to appropriately package the data elements and send them to the intended location, the CE Device (6) can additionally conduct its own analyses and processing for the purpose of alerting (or otherwise notifying) the consumer of certain states or conditions that might exist. For example this can be accomplished by loading software onto the CE Device which accomplishes these tasks using the resources of the CE Device.

The Network (7) is either a public or private network that can facilitate the flow of information from the Pod (1) to the appropriate Analysis and Reporting Service (8). It is obvious that the flow of data must be two-way as the Analysis and Reporting Service (8) may have information that needs to be communicated to both the CE Device (6) and the Pod (1).

The Analysis and Reporting Service (8) is the entity responsible to collect the empirical data received from multiple Pods (1), format the data as required, perform the necessary analyses, and then report the findings in an expeditious manner back to the intended consumers. In one embodiment the functions of the analysis and reporting service are accomplished by the CE Device itself.

In the end, the Analysis and Reporting Service (8) will be responsible to prepare and send its findings to consumers in ways that are secure, expeditious, user-friendly, helpful, and informative.

### Other Considerations

It is of the utmost importance that this entire system be secure. These data elements collected from a consumer are considered confidential information and cannot be shared with anyone else without the express consent from the consumer. Therefore, well-known security technologies such as SSL, AES-128 Encryption, Public Key Infrastructure, X.509 standards and protocols, RSA technologies and others can be used to verify the identity of the consumer and make sure the appropriate reports are sent back to the same consumer without any form of potential piracy or compromise.

The examples used above revolve around health-related risks. Alternatively, it is possible to collect information for other disciplines as well. For example, a consumer may be concerned about biological hazards that he or she may be exposed to. Using such an example, the consumer is not worried so much about his or her own vital statistics. Instead, the consumer is more concerned about the immediate environment. In this case, the system still works using the same basic principals. The data is collected by a Pod (1), the data is the organized appropriately and then sent on for analyses. The results of the analyses are then reported back to the consumer in a timely, efficient, and ultimately helpful manner.

What is claimed is:

1. An electronic device for rendering for display or audition stored digital audiovisual entertainment content, comprising: (a) data storage which may be either a rotational magnetic or optical disk, solid state electronics, or both; (b) a display mechanism to convey instructions to users, and make decision choices available for selection; (c) a data or selection entry mechanism such as a keyboard, switches, or touch sensitive areas for conveying information to the device; (d) an internal power source; (e) a variety of connectors used to interface the device to external devices; (f) an electronic computational module for processing computer software instructions; (g) a variety of electronic modules used to acquire and convert signals supplied to the device through connectors (1.e) or through radio frequency reception means to a form of digital data that can be stored on the internal data storage (1.a); (h) a variety of electronic modules used to convert data supplied to the device to render audio and visual entertainment content; (i) operational computer software supplied to acquire, convert, format, and store data, interact with human operators via display mechanism (1b.), interact with external electronic devices and processes contained within those devices and to cause the stored data to be transferred to those devices and, in turn, to receive instructions, alternative or additional operational computer software, or digital audiovisual entertainment content; (j) companion electronic devices intended to transmit and receive data to the portable device and to subsequently communicate with external devices and systems via bidirectional electronic, radio frequency, or optical transmission means.

2. The secondary purpose of the devices, software, and configuration of claim 1, which is extended by a variety of devices and software to be described below, is to convert, format, store, and subsequently transmit acquired data from external analog and digital sensor devices.

3. The acquired data referred to in claim 2 is human physiological/biomedical sensor data.

4. The acquired data referred to in claim 2 is nonhuman physiological/biomedical sensor data.

5. The acquired data referred to in claim 2 is physical sensor data.

6. The acquired data referred to in claim 2 is geophysical sensor data.

7. The acquired data referred to in claim 2 is adapted upon acquisition by specialized external devices and software designed to accommodate conversion from external sources to a format suitable for subsequent storage and transmission processes.

8. The data referred to in claim 7 is transmitted using a Set Top Box or Personal Computer that mates with the device of claim 2.

9. The data referred to in claim 7 is submitted via Cable Television, Internet Protocol Television (IPTV), or Digital Subscriber Line (DSL) services.

10. The data referred to in claim 7 is encrypted to prevent revelation to unauthorized interceptors.

11. The key for encryption of the data referred to in claim 10 is supplied by each individual Remote External Analysis Service of claim 12.

12. The key referred to in claim 11 is derived by a cryptographic process that positively and uniquely identifies the device referred to in claim 2.

13. The data referred to in claim 7 is submitted to Remote External Analysis Services that specialize in computationally intensive data processing to obtain specialized indications from the data.

14. Subsequent retransmission of the data or indications referred to in claim 13 is via the Internet.

15. The data indications in claim 13 are used to prescribe: (a) medications; (b) procedures; (c) processes; (d) subsequent tests and software to be loaded into the device of claim 2; (e) diagnostic and analytical conclusions to be provided to other cognizant individuals and organizations.
